(19) United States
(12) Patent Application Publication
(10) Pub. No.: US 2011/0190485 A1
(43) Pub. Date: Aug. 4, 2011

(54) SOLID-SUPPORTED ETHER DISULFIDES

(75) Inventor: David Allen Berry, Ann Arbor, MI (US)

(73) Assignee: Berry and Associates, Inc., Dexter, MI (US)

(21) Appl. No.: 12/698,591

(22) Filed: Feb. 2, 2010

(51) Int. Cl. C07H 21/00; C07C 69/716

(52) U.S. Cl. 536/25.6; 560/174; 536/22.1

(57) ABSTRACT

The present invention provides for compounds of Formulas I-III, and salts thereof,

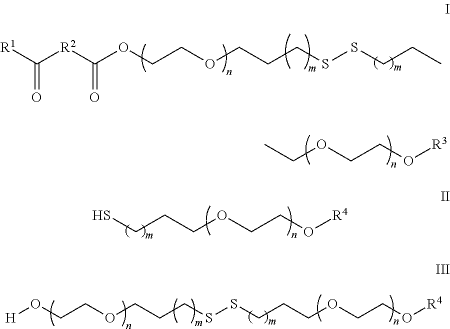

wherein n, m, R¹, R², R³, and R⁴ have any of the values defined there for in the specification. The compounds of formula I may be used in the synthesis of oligonucleotides such as compounds of formula II (bearing a 3′-thiol) or formula III (bearing a 3′-disulfide).

### BACKGROUND

The solid-supported synthesis of oligonucleotides is well established technology that is known to those skilled in the art of DNA and RNA chemistry. The solid-supported format facilitates the preparation of oligonucleotides with automated synthesizers. Many of the automated oligonucleotide synthesis protocols in the field employ one of the three solid supports depicted as follows:

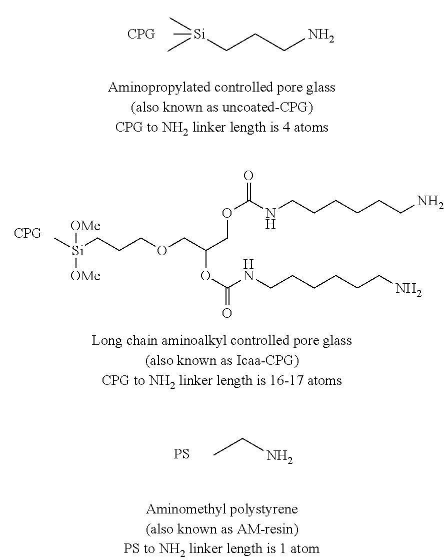

Controlled pore glass, also known as CPG, is the most common underivatized solid support. The surface of the glass is often modified either to contain amino-propyl groups or long chain aminoalkyl groups. The long chain aminoalkyl (lcaa) modification depicted has two advantages. First, it doubles the number of amine connection sites. Second, the distance between the amine connection sites and the solid glass surface is increased, thereby facilitating the chemical reactions of oligonucleotide synthesis. Polystyrene is a solid support that is used much less frequently for oligonucleotide synthesis. It is typically functionalized with aminomethyl groups as connection sites. The synthesis support most frequently employed in oligonucleotide synthesis is lcaa-CPG.

In synthesizing oligonucleotides, it can be useful to include a thiol or a disulfide as a modification to the oligonucleotide. These sulfur containing groups, not present in natural oligonucleotides, enable attachment of the oligonucleotide to surfaces or to other molecules of interest. For example Plutowski, et al. (Organic Letters, 2007, 9 (11), 2187-2190) describe the use of oligonucleotides, modified with thiols to the surface of gold nanoparticles. The added thiol or disulfide group can be placed at either the 5′-terminus of the oligo, the 3′-terminus of the oligo, or at some internal location in the oligo sequence. When the modification is to occur at the 3′-terminus of the oligo, a typical strategy is to link one end of a symmetrical alkyl disulfide to the solid support via an amide bond to the terminal amino group of the long chain amino alkyl moiety. The oligonucleotide chain is then extended from the other end of the disulfide as depicted in the following Scheme:

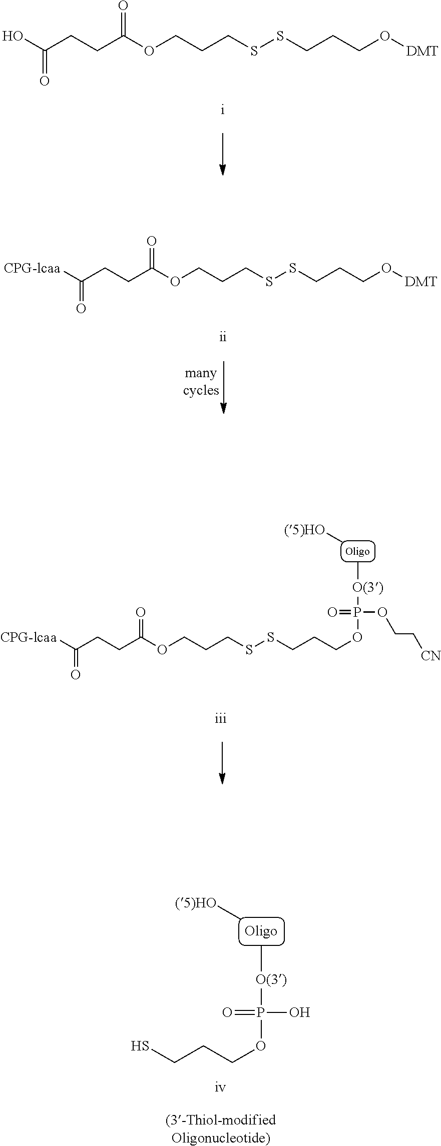

The hemisuccinate (i) is coupled to the amino groups on CPG-lcaa to afford ii. The solid supported oligonucleotide (iii) is synthesized via cycles of nucleotide incorporation, known to those skilled in the art. Deprotection and reductive cleavage from the support affords the free 3′-thiol-modified oligonucleotide (iv).

While the n-propylthiol modification shown in scheme above is reasonable for some applications, others may require a longer version such as n-hexylthiol. This creates an unexpected difficulty when a support such as lcaa-CPG support is used. The combination of C6-disulfide and lcaa-CPG generate a solid support that is markedly inferior for oligo synthesis at least in terms of the yield and purity of 3′-thiol-modified oligonucleotide. These problems are intensified as the length of the desired oligonucleotide increases. The use of C6-disulfide is somewhat improved by combination with a CPG that has a shorter linker. For example, attachment of the C6-disulfide to uncoated-CPG somewhat improves the yield and purity of 3′-(C6-thiol)-modified oligonucleotide.

Maximum oligo synthesis length is a global measure of the performance of the various 3′-thiol-modifier and solid support combinations. In all oligonucleotide syntheses, the cycles of reactions used to incorporate each nucleotide are very efficient but less than perfect. Ideally the coupling efficiency for a single nucleotide exceeds 99%. Even at this high efficiency, the yield and purity of the full length oligo suffers with each additional nucleotide. Eventually the impurities resulting from incomplete coupling grow to a point that the full length oligo is difficult to isolate and is produced in low yield.

There are research and diagnostic utilities for oligos that contain a 3′-thiol or 3′-disulfide modifications wherein the thiol or disulfide group is six or more atoms distant from the oligo. The current state of the art is that combination of well established solid supports with C6-disulfide limits the length of oligo that is synthetically feasible. Disulfides of equal to or greater length than C6-disulfide, in particular those which would be compatible with well accepted synthesis supports and still perform well in the synthesis of long oligos (10-100 nucleotides in length), would fulfill a need in the art.

### BRIEF SUMMARY

In one aspect, the present invention provides for compounds of Formula I, or a salt thereof:

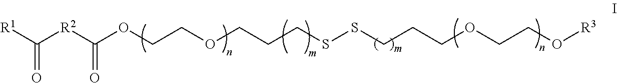

wherein: R¹ is OH, Cl, O-(succinimid-1-yl), O-(2,3,4,5,6-pentafluorophen-1-yl), O-(phthalimid-2-yl), O-(1,2,3-benzotriazo-1-yl), lcaa-CPG, uncoated-CPG, AM-resin, or a solid support suitable for oligonucleotide synthesis; R² is CH₂CH₂, CH₂CH₂CH₂, CF₂CF₂, CH₂OCH₂, 1,2-phenylene, 1,3-phenylene, 1,4-phenylene, 1,2-bis(CH₂O)benzene, 1,3-bis(CH₂O)benzene, or 1,4-bis(CH₂O)benzene; R³ is H, DMT, MMT, Si(Me)₂(t-Bu), Ski-PO₃, or Boc; n is an integer that is from 1 to 6; and m is either 0 or is an integer that is from 1 to 4. In particular embodiments, R¹ is OH or lcaa-CPG. In other embodiments, R² is CH₂CH₂ or CH₂OCH₂. In yet other embodiments, R³ is H or DMT. In certain embodiments, n is 1 or 2. In other embodiments, m is 0 or 1.

In another aspect, the present invention provides for compounds of formulas II and III, or a salt thereof:

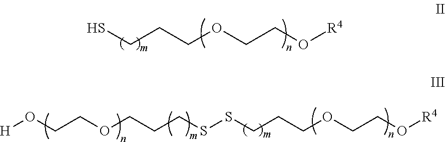

wherein:
R⁴ is an oligonucleotide of between 2 to 110 nucleotide residues;
n is an integer that is from 1 to 6; and
m is either 0 or an integer that is from 1 to 4. In particular embodiments, n is 1 or 2. In other embodiments, m is 0 or 1. In yet other embodiments, n is 1 or 2, and m is 0 or 1. In certain embodiments, R⁴ is an oligonucleotide of between 2 to 100 nucleotide residues. In particular embodiments, R⁴ is an oligonucleotide of between 10-100 nucleotide residues. In certain embodiments, R⁴ is an oligonucleotide of between 10 to 75 nucleotide residues.

### DEFINITIONS

“AM Resin” means aminomethyl-poly(styrene-divinylbenzene), an insoluble polymer that is prepared from 80-99% styrene and 1-20% divinylbenzene, which is subsequently chemically modified to attach aminomethyl groups such that the nitrogen content is 20 to 200 micromoles per gram of polymer.

“Boc” means tert-butyloxycarbonyl.

“CPG” means controlled pore glass.

“DCC” means N,N′-dicyclohexylcarbodiimide.

“DCM” means dichloromethane.

“DIC” means N,N′-diisopropylcarbodiimide.

“DMAP” means 4-dimethylaminopyridine.

“DMT” means bis(4-methoxyphenyl)(phenyl)methyl, which is also known as dimethoxytrityl.

“DNA” means (2′-deoxyribo)nucleic acid.

“EDAC.HCl” means N-ethyl,N′-dimethylaminoethylcarbodiimide hydrochloride.

“EtOAc” means ethyl acetate.

“Fmoc” means fluorenylmethyloxycarbonyl.

“HOBT” means 1-hydroxybenzotriazole.

“i-Bu” means isobutyl, 2-methylpropyl, or —CH₂CH(CH₃)₂.

“i-Pr” means isopropyl, 2-propyl, or —CH(CH₃)₂.

“iPr₂NEt” means Hünig's base, which is also known as N,N-diisopropylethylamine

“Me” means methyl or CH₃.

“MeCN” means acetonitrile.

“MeOH” means methanol.

“MMT” means bis(phenyl)(4-methoxyphenyl)methyl, also known as monomethoxytrityl.

“Mono-salt” means only one salt within a single compound. For example with a tricarboxylic acid compound, the addition of one molar equivalent of a base creates a mono-salt.

“Oligo” means oligonucleotide or a segment of DNA or RNA.

“Oligonucleotide” meansa sequence of two or more naturally occurring or non-naturally occurring nucleotide residues. “PCR” means polymerase chain reaction.

“Ph” means phenyl or C₆H₅.

“PS” means poly(styrene-divinylbenzene).

“PYBOP” means (benzotriazo-1-yloxy)tripyrrolidinophosphonium hexafluorophosphate

“RNA” means ribonucleic acid.

“t-Bu” means tertiary-butyl or C(CH₃)₃.

“THF” means tetrahydrofuran.

“TLC” means thin layer chromatography.

“Tr” means triphenylmethyl, also known as trityl.

### DETAILED DESCRIPTION

The compounds of Formula I may be used to effectively synthesize 3′-thiol-modified oligonucleotides that include a 6-atom or longer chain between the oligonucleotide and the thiol group. Oliognucleotides so prepared can be used in a variety of applications to bind to nucleotide sequences in diagnostic assays or for use in synthesizing oligonucleotides in amplification procedures such as PCR procedures.

Automated oligonucleotide synthesizers often employ nucleotides with a protecting group (e.g., DMT) that can be colorimetrically detected when liberated. For example, oligonucleotide synthesizers often measure the success of each nucleotide coupling by quantitatively monitoring the yellow color produced by cleavage of the DMT protecting group from the 5′-position of the most recently coupled nucleotide. Typically, a slight decline in the amount of yellow color produced with each additional nucleotide is observed. It is frequently observed that once the yellow color drops below a critical threshold the slope of decay increases more rapidly, making the continued elongation of the oligo unproductive. The critical threshold tends to be when the amount of yellow color is about 70% of the maximum color observed at the beginning of the oligonucleotide synthesis. Hence, the maximum oligo synthesis length is generally defined as the length that the oligo has reached when the yellow color observed upon DMT cleavage falls below the 70% level.

Table 1 summarizes the performance of different combinations of supports and disulfides in terms of maximum oligo synthesis length. The combination of a long linker and a C6-disulfide (compound 3) seriously impairs oligo synthesis, providing a maximum synthesis length of only 9 nucleotides. Shortening of either the CPG linker length or the disulfide alkyl chain improves synthesis performance. The combination of uncoated-CPG and C6-disulfide (compound 2) gives a maximum synthesis length of 45 nucleotides. The combination of lcaa-CPG and C3-disulfide (compound 1) gives a maximum synthesis length greater than 75 nucleotides (the limit of the experiment). The shorter disulfide in compound 1 offers the greatest performance in terms of synthesis length compared to compound 2 or 3.

TABLE 1
3′ Thiol-modifier supports and their performance in oligonucleotide synthesis

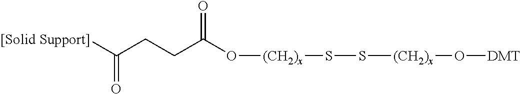

| Compound | | CPG Linker length | | Maximum Oligo |
|---|---|---|---|---|
| Number | Solid Support | (# of atoms) | x | Synthesis Length |
| 1 | lcaa-CPG | 16-17 | 3 | >75 |
| 2 | uncoated-CPG | 4 | 6 | 45 |
| 3 | lcaa-CPG | 16-17 | 6 | 9 |

Compounds of formula I may be used prepared as follows.

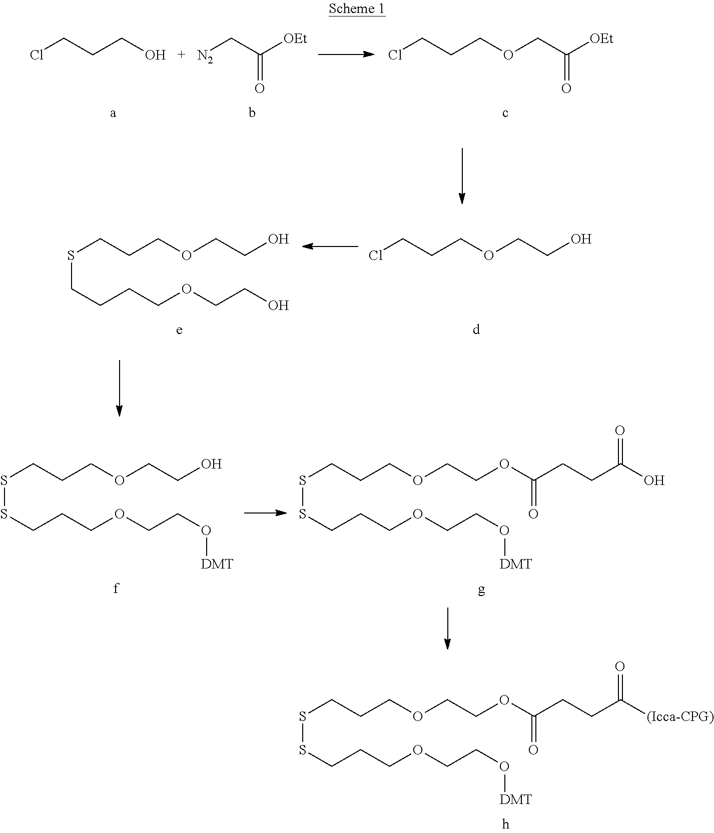

Although Scheme 1 depicts the synthesis of compound h, it may be generally be modified to synthesize compounds of formula I by using analogous starting materials and intermediates. In Scheme 1, compound a (3-chloropropan-1-ol) may be reacted in a solvent such as DCM, 1,2-dichloroethane, toluene, or benzene with a diazo ester b (e.g., ethyl diazoacetate) and a Lewis Acid such as boron trifluoride-etherate or TiCl₄ to generate compound c (e.g., ethyl 2-(3-chloropropoxy)acetate). The compound c can then be reduced in an anhydrous solvent such as THF using LiAlH₄, (i-Bu)₂AlH₃, or AlH₃ to form compound d (e.g., 2-(3-chloropropoxy)ethanol). The compound d may then be reacted in a mixture with thiourea and water at reflux to provide compound e (e.g., 3,12-dioxa-7,8-dithiatetradecane-1,14-diol).

The compound e may then contacted with an anhydrous solvent such as anhydrous DCM or 1,2-dichloroethane, under a nitrogen atmosphere with an anhydrous tertiary amine such as triethylamine or diisopropylethylamine in the presence of a suitable protecting group reagent such as DMT-Cl to generate compound f (14-(dimethoxytrityl)oxy-3,12-dioxa-7,8-dithiatetradecane-1-ol). Examples of other suitable protecting groups include, but are not limited to, MMT, Si(Me)₂(t-Bu), Si(i-Pr)₃, or Boc.

The compound f in a solvent such as anhydrous pyridine or a mixture of anhydrous pyridine and another solvent such as DCM or THF may then be treated succinic anhydride. DMAP may optionally be added to obtain a faster reaction. The resulting solution may then be stirred from 2 to 24 hours at room temperature to yield compound g (1-(dimethoxytrityl)oxy-7,8,dithia-16-oxo-3,12,15-trioxanonadecan-19-oic acid).

Compounds such as compound g may be coupled with any solid support suitable for oligonucleotide synthesis through its terminal carboxylic acid functionality. Examples of suitable solid supports include, but are not limited to, lcaa-CPG, uncoated-CPG, and AM-resin. For example, compound g may be contacted with lcaa-CPG in anhydrous DCM in the presence of amide bond coupling reagents, which are well known to one skilled in the art of organic synthesis, such as PYBOP+iPr₂NEt, DCC+HOBT, DIC+HOBT, or EDAC.HCl+HOBT to afford compound h. Capping of remaining amino groups by treatment with acetic anhydride and a suitable base such as pyridine, collidine, DMAP, or combinations thereof may optionally be carried out if the reaction to convert g to h does not go to 100% completion. Compounds such as compound h may be used directly in an automated oligonucleotide synthesizer according to methods well known to those skilled in the art of automated oligonucleotide synthesis.

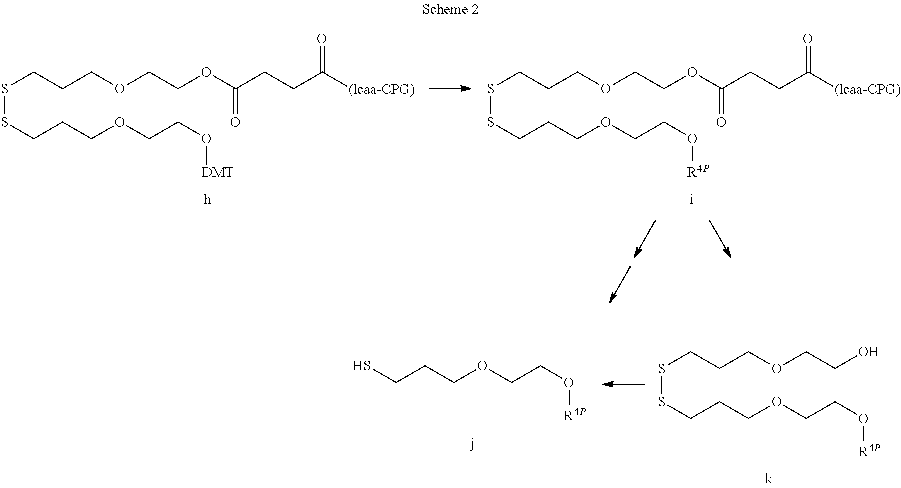

Compounds such as compound h may be used to carry out solid-supported oligonucleotide synthesis (Scheme 2) to provide for a compound such as compound i, where R^{4P} is a protected form of an oligonucleotide. The compound j, where R⁴ is an oligonucleotide, may be liberated from compound i in two steps by first reducing the disulfide with dithiothreitol or a comparable disulfide reducing agent, and second removing the oligo protecting groups by treatment with aqueous ammonium hydroxide, an aqueous mixture of ammonium hydroxide and methylamine, or methanolic potassium carbonate. Alternatively, compound j may be liberated from compound i by carrying out these two steps in the reverse order. That is to say compound k is first produced by treatment with aqueous ammonium hydroxide, an aqueous mixture of ammonium hydroxide and methylamine, or methanolic potassium carbonate, then compound j is produced by reduction of the disulfide of compound k with dithiothreitol. The methods and conditions for thiol-modified oligonucleotide deprotection and cleavage from solid supports are well known to those skilled in the art of oligonucleotide synthesis.

In certain embodiments, the compounds of the present invention, e.g., compounds of formulas I-III may be capable of forming base addition salts. Acceptable base addition salts may be formed with metals, such as alkali and alkaline earth metal hydroxides, or with organic tertiary amines. Examples of metals used as cations are calcium, magnesium, potassium, sodium, lithium, and the like. Examples of suitable tertiary amines include triethylamine, N,N-di(isopropyl)-ethylamine, N-methylmorpholine, N-methylpiperidine, N-methylpyrrolidine, and the like, and aromatic amines, such as pyridine, 2,6-dimethylpyridine, 2,4,6-trimethylpyridine, 4-(dimethylamino)pyridine, and the like.

### EXAMPLES

### Example 1

### Ethyl 2-(3-chloropropoxy)acetate (6)

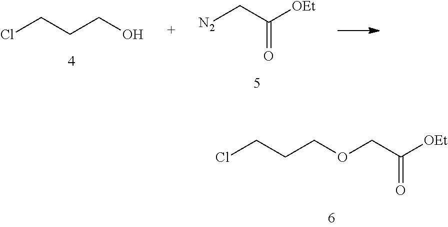

A solution of 3-chloropropan-1-ol (4, 64 g, 0.68 Mol) in DCM (0.68 L) was cooled with an ice/MeOH bath. Ethyl diazoacetate (5, 77 g, 0.67 Mol) was added and mixed well before adding boron trifluoride-etherate (1.0 g, 7.0 mMol). The latter addition started nitrogen gas evolution from the reaction. The cold bath was removed and the temperature was allowed to rise to 37′C where it was maintained until gas evolution ceased. Solvent was evaporated from the reaction mixture at reduced pressure to give an orange oil. Vacuum distillation using an 8-inch tall Vigreux column prior to the condenser afforded purified 6 (78.8 g, 65%). ¹H-NMR (CDCl₃, δ): 4.21 (q, 2H), 4.07 (s, 2H), 3.67 (m, 4H), 2.06 (p, 2H), 1.28 (t, 3H).

### Example 2

### 2-(3-chloropropoxy)ethanol (7)

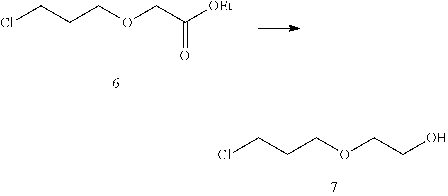

LiAlH₄ (11.9 g, 0.31 Mol) was added to a stirring solution of anhydrous THF (2 L). The resulting mixture was stirred under an atmosphere of dry nitrogen while cooling with an ice/MeOH bath. 6 (78.3 g, 0.44 Mol) was added and the cold bath was removed, stirring at room temperature under nitrogen overnight. The excess LiAlH₄ was quenched by careful addition of acetone (16 g) and the reaction was stirred one hour at room temperature. The resulting thickened mixture was cooled to 5° C. and 10% aqueous H₂SO₄ was added gradually to afford a gray slurry of solid. The solid was removed by filtration through a pad of CELITE® (diatomaceous earth), rinsing with THF (1 L). The combined filtrate and rinse were concentrated at reduced pressure. Vacuum distillation using a 6-inch tall Vigreux column prior to the condenser afforded purified 7 (49.55 g, 82%). ¹H-NMR (CDCl₃, δ): 3.70 (t, 2H), 3.61 (p, 4H), 3.53 (t, 2H), 2.34 (br, 1), 2.01 (p, 2H).

### Example 3

### 3,12-dioxa-7,8-dithiatetradecane-1,14-diol (8)

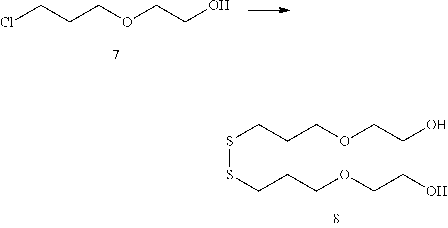

A mixture of 7 (49.5 g, 0.357 Mol), thiourea (27.2 g, 0.357 Mol), and water (396 mL) was heated at reflux overnight. The mixture was then cooled to 5° C. and treated with a single charge of NaBO₃.4H₂O (57.7 g, 0.375 Mol), followed by gradual addition of 2M NaOH (268 mL, 0.536 Mol) over 15 minutes. The cold bath was removed and the reaction mixture was stirred as it warmed gradually to 31′C as a mildly exothermic reaction ensued. When the mixture had cooled to room temperature again, it was extracted with DCM (4×500 mL). The combined DCM extracts were dried over Na₂SO₄, filtered, and concentrated at reduced pressure. The resulting oil was placed under vacuum at room temperature until a constant weight was obtained, affording 8 (37 g, 76.8%) that is sufficiently pure for use in Example 4. MS (AP+) 271 (M+H), 293 (M+Na), 309 (M+K).

### Example 4

### 14-(Dimethoxytrityl)oxy-3,12-dioxa-7,8-dithiatetradecane-1-ol (9)

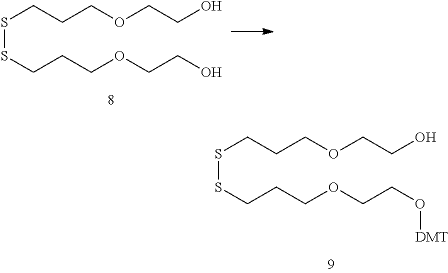

A solution of 8 (6.14 g, 22.7 mMol) in anhydrous DCM (35 mL) was placed under an atmosphere of dry nitrogen. Anhydrous triethylamine (3.8 mL, 27.2 mMol) was added. The resulting solution was cooled on an ice bath for 15 minutes. A solution of DMT-Cl (7.69 g, 25 mMol) in anhydrous DCM (55 mL) was added dropwise over 50 minutes then the cold bath was removed, stirring one hour further. The resulting solution was washed with water (2×50 mL), washed with brine (25 mL saturated NaCl plus 25 mL water), dried over Na₂SO₄, and filtered. The filtrate was concentrated at reduced pressure to give a yellow oil. Flash chromatography in silica gel, eluting with a gradient of 25-60% EtOAc in hexanes affords 9 (6.5 g, 50%) as a colorless oil upon evaporation of solvents. TLC (Silica gel on glass, eluted with 75 hexanes: 25 EtOAc) shows a single spot with R_{f}=0.28.

### Example 5

### 1-(Dimethoxytrityl)oxy-7,8,dithia-16-oxo-3,12,15-trioxanonadecan-19-oic acid (10)

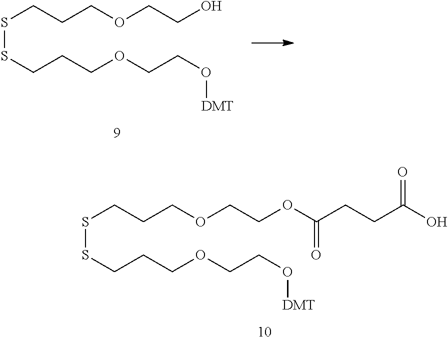

A solution of 9 (6.8 g, 11.9 mMol) in anhydrous pyridine (15 mL) was treated with DMAP (0.24 g, 2 mMol) followed by succinic anhydride (1.78 g, 17.8 mMol). The resulting solution was stirred overnight at room temperature. The reaction mixture was cooled on an ice bath and then treated with water (1.1 mL, 61.1 mMol). The cold bath was removed and the solution was allowed to warm to room temperature. The resulting solution was concentrated in vacuo to give a viscous yellow concentrate. The concentrate was dissolved in EtOAc (400 mL) and this solution was washed with water (2×400 mL). The EtOAc solution was then concentrated in vacuo to give a viscous yellow concentrate. Flash chromatography on silica gel, eluting with a gradient of 2-10% MeOH in DCM affords purified 10 (6.3 g, 78.7%) of a colorless gum. TLC (Silica gel on glass, eluted with 90 CHCl₃: 10 MeOH) shows a single spot with R_{f}=0.62.

### Example 6

### 1-(Dimethoxytrityl)oxy-7,8,dithia-16-oxo-3,12,15-trioxanonadecan-19-oyl-[lcaa-CPG] (11)

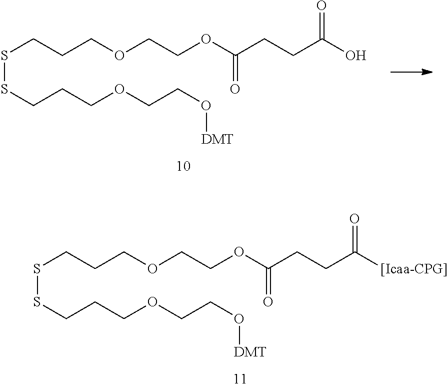

Dry lcaa-CPG (1.0 g, 91 μMol) was placed in a solid phase shake flask. Anhydrous DCM (6 mL) and anhydrous iPr₂NEt (0.16 mL) were added and the resulting slurry was shaken for 2 minutes. The bottom stopcock of the flask was opened and the liquid phase was expelled under a positive pressure of dry nitrogen gas. A freshly prepared solution composed of 10 (61 mg, 91 μMol), anhydrous DCM (4 mL), iPr₂NEt (0.16 mL, 91 μMol), and PYBOP (47, 91 μMol) was added and the tightly capped flask was shaken at room temperature overnight. The liquid phase was expelled with dry nitrogen and then the solid was successively washed with DCM (2×6 mL), pyridine (2×6 mL), DCM (2×6 mL), pyridine (2×6 mL) and DCM (5×6 mL). The solid was dried under a stream of dry nitrogen then transferred to a vial and further dried under a vacuum at room temperature. This solid was further treated with a freshly prepared mixture of THF (4 mL), 2,4,6-collidine (0.12 mL) and acetic anhydride (43 μL), shaking at room temperature for 1.5 hours. It was then washed and dried as above to afford 11. A sample was subjected to a solution of toluenesulfonic acid in acetonitrile. The resulting yellow DMT cation that was liberated from the solid into solution was quantified by UV absorbance at 498 nM, indicating 42+/−4 μMol per gram of 11.

### Example 7

### Comparison of 3 and 11 in the synthesis of (Poly-T)-O(CH₂)₆SS(CH₂)₆C(═O)CH₂CH₂C(═O)-(lcaa-CPG) and (Poly-T)-O(CH₂)₂O(CH₂)₃SS(CH₂)₃O(CH₂)₂OC(═O)CH₂CH₂C(═O)-(lcaa-CPG)

Using a Millipore Expedite (8900 series) nucleic acid synthesis system (Billerica, Mass.), freshly prepared reagent solutions were installed as follows were installed in the reagent bottles as follows:
Wash A—anhydrous acetonitrile
Deblock—3% Trichloroacetic acid in anhydrous dichloromethane
Oxidizer—0.02M iodine in tetrahydrofuran/water/pyridine
Capping reagent A—acetic anhydride/anhydrous tetrahydrofuran
Capping reagent B—16% 1-methylimidazole in anhydrous tetrahydrofuran/pyridine
Wash reagent—anhydrous acetonitrile
Activator—0.25M 5-ethylthiotetrazole in anhydrous acetonitrile
Amidites: Thymidine-CEP (0.067M solutions in anhydrous acetonitrile)
The reagent lines were purged and pumps primed. Two 200 nM synthesis columns, one containing 3 (Table 1) and the other containing 11 (EXAMPLE 6) were installed. The instrument run parameters were then set as follows for both Column-1 and Column-2:
Sequence—T200 (denoting an oligo that contains 200 Thymidines)
Protocol—CYCLE T (a 23 step protocol for reagent additions, reaction times, and washes known to be optimized for each coupling of Thymidine-CEP, as provided in the synthesizer software)
Final DMT—On (The DMT of the X residue is not subjected to Deblock solution)

(Poly-T)-O(CH₂)₆SS(CH₂)₆C(═O)CH₂CH₂C(═O)-(lcaa-CPG) was synthesized in Column 1 using CYCLE T conditions for each thymidine incorporation. The output of the colorimetric monitoring of each DMT deblock step was recorded by the synthesizer's computer. After incorporation of 9 thymidines, the DMT color signal was reduced to 70% of the maximum value seen at the beginning of the synthetic run and continues to decline as the oligo synthesis continues. Therefore the maximum oligo synthesis length is only 9 nucleotides.

(Poly-T)-O(CH₂)₂O(CH₂)₃SS(CH₂)₃O(CH₂)₂OC(═O)CH₂CH₂C(═O)-(lcaa-CPG) was synthesized in Column 2 using the identical protocol to Column 1. The output of the colorimetric monitoring of each DMT deblock step was recorded by the synthesizer's computer. The 70% threshold of DMT color signal was reached at 104 nucleotides and continued to decline thereafter. Therefore compound 11, a compound of formula I, shows clearly superior performance in oligonucleotide synthesis compared to the existing state of the art, as exemplified by compound 3.

What is claimed is:

1. A compound of Formula I, or a salt thereof: 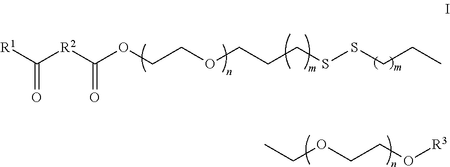
wherein:
R¹ is OH, Cl, O-(succinimid-1-yl), O-(2,3,4,5,6-pentafluorophen-1-yl), O-(phthalimid-2-yl), O-(1,2,3-benzotriazo-1-yl), lcaa-CPG, uncoated-CPG, AM-resin, or a solid support suitable for oligonucleotide synthesis;
R² is CH₂CH₂, CH₂CH₂CH₂, CF₂CF₂, CH₂OCH₂, 1,2-phenylene, 1,3-phenylene, 1,4-phenylene, 1,2-bis(CH₂O)benzene, 1,3-bis(CH₂O)benzene, or 1,4-bis(CH₂O)benzene;
R³ is H, DMT, MMT, Si(Me)₂(t-Bu), Si(i-Pr)₃, or Boc;
n is an integer that is from 1 to 6; and
m is either 0 or is an integer that is from 1 to 4.

2. The compound of claim 1, wherein R¹ is OH or lcaa-CPG.

3. The compound of claim 2, wherein R² is CH₂CH₂ or CH₂OCH₂.

4. The compound of claim 2, wherein R³ is H or DMT.

5. The compound of claim 1, wherein n is 1 or 2.

6. The compound of claim 5, wherein m is 0 or 1.

7. The compound of claim 6, wherein R¹ is OH or lcaa-CPG and R² is CH₂CH₂ or CH₂OCH₂.

8. The compound of claim 7, wherein where R³ is H or DMT, n is 1 or 2, and m is 0 or 1.

9. The compound of claim 8, wherein R¹ is OH or lcaa-CPG, R² is CH₂CH₂ or CH₂OCH₂, R³ is H or DMT, n is 1, and m is 1.

10. The compound of claim 1, wherein said compound is selected from the group consisting of:
1-(Dimethoxytrityl)oxy-7,8,dithia-16-oxo-3,12,15-trioxanonadecan-19-oic acid; and
1-(Dimethoxytrityl)oxy-7,8,dithia-16-oxo-3,12,15-trioxanonadecan-19-oyl-[lcaa-CPG].

11. A compound of Formula II or III, or a salt thereof: 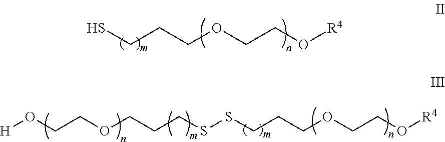
wherein:
R⁴ is an oligonucleotide of between 2 to 110 nucleotide residues;
n is an integer that is from 1 to 6; and
m is either 0 or an integer that is from 1 to 4.

12. The compound of claim 11, wherein n is 1 or 2.

13. The compound of claim 12, wherein m is 0 or 1.

14. The compound of claim 13, wherein n is 1 or 2, and m is 0 or 1.

15. The compound of claim 14, wherein R⁴ is an oligonucleotide of between 10 to 100 nucleotide residues.
